(19) United States
(12) Patent Application Publication
(10) Pub. No.: US 2003/0228276 A1
(43) Pub. Date: Dec. 11, 2003

(54) Neuroprotective and neurogenerative effects of the long-term expression of TNF alpha in the substantia nigra and a new animal model for Parkinson's disease

(76) Inventors: Fernando Juan Pitossi, Buenos Aires (AR); Ulrich Lothar Maria Eisel, Waiblingen (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(21) Appl. No.: 10/410,012

(22) Filed: Apr. 9, 2003

Related U.S. Application Data
(60) Provisional application No. 60/370,974, filed on Apr. 9, 2002.

(51) Int. Cl. A61K 38/19; A61K 38/20; A61K 38/44

(52) U.S. Cl. 424/85.1; 424/85.2; 424/94.4; 514/12

(57) ABSTRACT

Methods for treating a neurodegenerative disease in a mammal, specifically methods for treating Parkinson's disease. The treatment is based on administering an effective amount of a TNF-related molecules and/or TNF down regulating molecules, acting preferably in the substantia nigra. The effects of TNF over the substantia nigra are disclosed. Low concentration of TNF provides a neuroprotective activity and high concentration of TNF provides a neurodegenerative effect on the mammal. The method is based on a treatment with TNF wherein the concentration of TNF in the patients affected by the Parkinson's disease is regulated. In addition, a treatment in which molecules activate a survival signal into neurons via TNFR 2 receptors is provided. A new animal model for Parkinson's disease studies is also provided, wherein the adenovirus vector, the CRE/loxP and transgenic knock-in mice are combined. The transgenic knock in mouse has a transgene comprising a DNA sequence that encodes mTNF in operable linkage with the endogenous engrailed-1 promoter; and a loxP interference cassette downstream of the transgene, wherein the mTNF coding sequence is expressed at low levels in the substantia nigra, and mimics Parkinson's pathological conditions.

This is a nonprovisional of U.S. Ser. No. 60/370974, filed Apr. 9, 2002.

### BACKGROUND OF THE INVENTION

1. Field of the Invention

The invention relates to methods of treating neurodegenerative diseases in mammals, more particularly in human beings, in need of such treatment, wherein the treatment is based in the regulation of the TNF activity in the patient. More particularly, the treatment comprises administering to the patient an effective amount of TNF signal transduction pathway compounds and/or TNF down regulating molecules. We disclose the effects of TNF over the substantia nigra, wherein such effects depend on the concentration of TNF in the substantia nigra. A low concentration of TNF provides a neuroprotective activity and a high concentration of TNF provides a neurodegenerative effect on the mammal. More specifically, the invention relates to a method of treating a patient affected by Parkinson's disease. A treatment with TNF is provided by the invention wherein the activity of TNF in patients affected by the Parkinson's disease is regulated. In addition, treatments in which molecules activate a survival signal into neurons via TNF receptor type 2 (TNFR 2) or inhibit a death signal from the TNF receptor type I is also provided.

The invention also relates to a new animal model for use in the studying of the Parkinson's disease, wherein the adenovirus vector, the CRE/loxP and transgenic knock-in mice are combined.

2. Description of the Related Art

Parkinson's Disease (PD) is a neurodegenerative, slow progressive disorder whose main pathological characteristic is the selective loss of dopaminergic neurons in the substantia nigra (s.n.) (Lang, A. E. & Lozano, A. M. The New England Journal Of Medicine 339: 1044-1053, 1998 and Beal, M. F. Nat Rev Neurosci 2: 325-334, 2001). The etiology of PD and the cause of the preferential vulnerability of neurons from this area remain unknown. A genetic component accounts for at least a percentage of PD cases. A main contribution of reactive oxygen or nitrogen species and free radicals to dopaminergic cell death in the s.n. has been described (Beal, M. F. Nat Rev Neurosci 2, 325-334, 2001 and Hirsch, E. C. & Hunot, S. Trends Pharmacol Sci 21: 163-165, 2000). Activation of microglial cells and induction of pro-inflammatory cytokines such as tumor necrosis factor α (TNF) and interleukin-1 in the s.n. have been observed in animal models and post-morten tissues of PD patients (Hirsch, E. C. & Hunot, S. Trends Pharmacol Sci 21: 163-165, 2000 and Nagatsu, T., et. al. Neural Transm Suppl , 277-290, 2000). However, it is not clear whether this microglial activation could be functional to remove cellular debris after neuronal death or could be propagating, slowering or even triggering the death of dopaminergic neurons (Hirsch, E. C. & Hunot, S. Trends Pharmacol Sci 21: 163-165, 2000). In addition, the s.n. is the brain region with the highest density of microglial cells (Lawson, L. J., et. al., Neuroscience 39: 151-170, 1990) and a differential susceptibility of this region to the toxic effects of endotoxin, a cytokine inducer, has been described (Kim, W. G. et al. J Neurosci 20: 6309-6316, 2000).. Furthermore, an increased susceptibility to inflammation with aging has also been suggested, pointing to a differential role of cytokines in diseases of the elderly such as most neurodegenerative disorders (Chung, H. Y., et. al, Ann N Y Acad Sci 928: 327-335, 2001).

In general, the role of the mediators of the innate immunity, such as TNF, on neurodegeneration and neuroprotection is controversial (Nguyen, M., et. al., Nature Neuroscience reviews 3: 216-227, 2002). This cytokine could have opposite effects on neuronal survival. It has been assumed that this opposite effects of TNF on the same animal model or type of neuron was dependent on its time of expression, the acute vs. chronic TNF administration, or the presence of additional factors such as reactive oxygen species (Shohami, E., et. al., Cytokine Growth Factor Rev 10: 119-130, 1999 and Loddick, S. A. & Rothwell, N. J. Proc Natl Acad Sci USA 96: 9449-9451, 1999). TNF expression has been associated not only to PD, but to Alzheimer's disease, cerebral ischemia, AIDS dementia and Multiple Sclerosis as well (Venters, H. D., et. al., Trends Neurosci 23: 175-180, 2000). A plethora of evidence show both neuroprotective and neurodegenerative effects of TNF also in these diseases, although is has been primarily associated with the pathological condition (Stoll, G., et. al., J Neural Transm Suppl 59: 81-89, 2000). Therefore, the need exists of providing an animal model or system to allow the long-term expression of the desired molecule in discrete areas of the SNC of adult animals, such as the s.n., has hampered the answering of many of these issues. A number of transgenic animal models are known and which models are helpful for studying human diseases, such as WO95/05466, to Anderton et al, wherein a transgenic animal model for the study of the Alzheimer's disease is disclosed. This publication discloses the use of cells and transgenic animals for testing new therapeutic agents that are useful for treating the Alzheimer's disease. Also publication WO01/13715 discloses an animal model for pathologies involving monoamine dysfunction in which the vesicular monoamine transporter (2) (VMAT2) gene has been manipulated. Such models are useful particularly in the studying of Parkinson's disease, schizophrenia and drug dependencies, as well as in assay methods for obtaining agents of therapeutic potential in such disorders. This model is only useful when the pathology involves monoamine dysfunction and not when the pathology is related to high or low TNF levels, particularly TNF levels in the substantia nigra.

The probable mechanism of action of TNF in mediating neuronal death in the s.n. could be several. One actual line of thinking is that TNF (and also IL-1) is not supposed to be neurotoxic per se, but that they interfere with an endogenous neuronal survival signal, such as IGF-1 in cerebellar granule cells (Loddick, S. A. & Rothwell, N. J. Proc Natl Acad Sci USA 96: 9449-9451, 1999; Venters, H. D., et. al., Trends Neurosci 23: 175-180, 2000 and Venters, H. D. et al. J Neuroimmunol 119: 151-165, 2001). On the other hand, upregulated TNF could also mediate cell death directly by activating the apoptotic pathways via its type I receptor. Indeed there is already evidence available that brains from PD patients have this apoptotic pathway activated (Hartmann, A. et al. J Neurosci 21: 2247-2255, 2001 and Hunot, S. et al. J Neurosci 19: 3440-3447, 1999). Another firm possibility to explain the TNF-neurotoxic effects is by the induction of iNOS (Liberatore, G. T. et al. Nat Med 5: 1403-1409, 1999). A positive loop of induction among CD23, NO and TNF together with IFN-γ leading to neuronal toxicity in the s.n. has been convincingly described (Hirsch, E. C. & Hunot, S. Trends Pharmacol Sci 21: 163-165, 2000 and Liberatore, G. T. et al. Nat Med 5: 1403-1409, 1999). A cooperative toxicity between TNF and reactive oxygen species has been described (Ginis, I. et al. Mol Med 6: 1028-1041, 2000). On the other hand, the fact that the region studied (s.n.) contains a specially high number of microglial cells that could account for a preferential susceptibility of this region to TNF-mediated effects could add to any of the molecular events described before Indeed, the s.n has been already shown to be more susceptible to the damaging effect of the administration of a cytokine-inducer (LPS) than other brain regions (Castano, A., et. al., J Neurochem 70: 1584-1592, 1998 and Kim, W. G. et al. J Neurosci 20: 6309-6316, 2000). A model of TNF-mediated neurodegeneration should be useful to test these possibilities.

Several compounds for inhibiting TNF are disclosed, for example those compounds disclosed in WO00/18409. In addition, patent documents WO 98/05783, WO 98/57936, U.S. Pat. No. 6,187,543 y U.S. Pat. No. 5,811,300 disclose several compounds that may be employed in regulating TNFα values. U.S. Pat. No. 6,204,052, to Abraham Bout et al. discloses an adenoviral vector with a deletion in the region E3 in a manner that the remaining region E3 reduces the response to TNF in virus-harboring mammal host cells. Said vector is employed for genetic therapy treatment to reduce the host response to TNF. Publication PCT WO 00/73481, to Burstein et al., discloses methods and compounds for reducing TNF levels in TNF associated disorders. Recombinant adeno-associated virus (rAAV) vectors encoding necrosis tumor antagonist, methods using these vectors to reduce levels of TNF in a mammal, and methods of using these rAAV vectors in palliating TNF-associated disorders, such as articular (joints) diseases, are also known. Publication PCT WO 00/18409, to Olmarker et al. discloses pharmaceutical compositions for the treatment of spinal disorders caused by the liberation of TNFα comprising an effective amount of a TNFα inhibitors, such as metalloproteinase inhibitors, tetracyclines, quinolonescorticosteroids, thalidomide, lazaroides and others.

The discovery of a surprisingly beneficial role of TNF has been recently shown for oligodendrocytes after a careful analysis on demyelinating diseases and in an animal model for retinal ischemia (Fontaine, V. et al. J. Neurosci. 22: 216, 2002; Kassiotis, G. & Kollias, G. J Exp Med 193: 427-434, 2001 and Arnett, H. A. et al. Nat Neurosci 4: 1116-1122, 2001). TNF was shown to accelerate the onset of EAE and induce oligodendrocyte apoptosis but it was also necessary for the regression of myelin-specific T cell activity in this animal model of multiple sclerosis (MS) (Kassiotis, G. & Kollias, G. J Exp Med 193: 427-434, 2001). In addition, TNF was described to have an accelerating effect on acute demyelination but to induce the proliferation of oligodendrocyte progenitors and remyelination (Arnett, H. A. et al. Nat Neurosci 4: 1116-1122, 2001). Anti-TNF therapies have failed to improve (or have even worsened) the symptoms of MS patients when TNF was previously supposed to be only deleterious for oligodendrocytes.

Studies on neuronal survival analysing only high levels of TNF using pharmacological means and transgenic animals or abolishing TNF expression by germline deletion have given controversial results (Shohami, E., et. al., Cytokine Growth Factor Rev 10: 119-130, 1999).

Finally, in the case of PD, an idea is emerging to visualise its future treatment as a multifactorial therapy (Grunblatt, E., et. al., Ann N Y Acad Sci 899: 262-273, 2000). In the light of our results, a careful examination of the levels of TNF in PD patients and experiments involving different TNF levels in already-lesioned animals will be required before designing a treatment that directly or indirectly modifies TNF levels. Thus, the invention discloses that upregulated levels of TNF result in a progressive loss of DA neurons in the s.n., however the constitutive expression of TNF is, unexpectedly, neuroprotective. A transgenic knock-in mouse as an animal model for Parkinson's disease is also provided.

### SUMMARY OF THE INVENTION

It is an object of the present invention to provide a method of treating a neurodegenerative disease, such as a Parkinson's disease, in a mammal in need of such treatment, the method comprising:

i) administering an effective amount of at least one TNF related molecule, or TNF modulating activity molecule as intracellular TNF-induced molecules, extracellular TNF-induced molecules, TNF analogous, TNF active muteins, TNF active fragments and TNFR 2 activating molecules. More specifically, the intracellular TNF-induced molecules may be NF-kB, MAP-K, PKB/Ak, JNK, p38, erks, ask1, gck, IKK, acidic and basic Sphingomyelinases, FAN or caspases; and the extracellular TNF-induced molecules may be MnSOD, GDNF, BDNF, TIP-b1, BDNF, IL-6, HLA, adhesion molecules. The TNF-related molecule may be TNFR 2 activating molecules. It is apparent for any expert in the art that any compound or molecule may be employed in the inventive treating method with the compound or molecule having a neuroprotective effect via regulation for low TNF expressions in the substantia nigra.

It is another object of the present invention to provide a method of treating a neurodegenerative disease such as a Parkinson's disease in a mammal in need of such treatment, the method comprising:

i) administering a nucleic acid for gene therapy, wherein said nucleic acid encodes TNF-related molecule or TNF modulating activity molecule selected from the group consisting of intracellular TNF-induced molecules, extracellular TNF-induced molecules, TNF analogous, TNF active muteins, TNF active fragments and TNFR 2 activating molecules. More specifically, the intracellular TNF-induced molecules may be NF-kB, MAP-K, PKB/Akt JNK, p38, erks, ask1, gck, IKK, acidic and basic Sphingomyelinases, FAN or caspases; and the extracellular TNF-induced molecules maybe MnSOD, GDNF, TIP-b1, BDNF, IL-6, HLA, adhesion molecules. In a preferred embodiment TNFR 2 activating molecules are employed. It is apparent for any expert in the art that any nucleic acid sequence may be employed in the inventive treating method with the sequence having a neuroprotective effect via regulation for low levels in the TNF expressions in the substantia nigra.

It is still another object of the present invention to provide a method of treating a neurodegenerative disease such as a Parkinson's disease in a mammal in need of such treatment, the method comprising:

i) determining the TNF levels and detecting an inflammatory disease in a group of mammals; ii) selecting the mammals having high TNF levels and/or and inflammatory disease; and iii) administering to the selected mammals a direct or indirect therapy that reduces the TNF levels, wherein the therapy that reduces the TNF levels comprises administering an effective amount of at least one molecule selected from the group consisting of TNF ineffective ligands, TNF down-expression regulating molecules, TNF down-biological activity regulating molecules, methaloproteases inhibitors, TNFR 1 inactivating molecules, TNFR 1 inhibitors and combinations thereof. More specifically the TNF muteins with altered capability to transduce signals after receptor binding , defective TNF receptor-binding domains, the TNF down-expression regulating molecules may comprise corticosteroids, anti-inflammatory cytokines such as RIP, Traf1 and TRAF2, Bid, soluble TNF receptors, phosphodiesterase inhibitors such as rolipram, RDP58 and prostanoids; molecules regulating; the TNF down-biological activity regulating molecules may comprise anti-TNF antibodies, Thalidomide, humanized anti-TNF antibody, geraniin, corilagin, epigallocatechin gallate, EtarneRcept, Infliximab, GM-CSF, histamine, Adalimumab, adrenoreceptor agonists, activators of vagal nerve activity, and antisense oligonucleotides; the methaloproteases inhibitors may comprise TACE inhibitors, N-terminal domain form of tissue inhibitor of metalloproteinases-3 (N-TIMP-3), CP-661,631, gamma-lactam hydroxamic acids, and E)-2(R)-[1(S)-(Hydroxycarbamoyl)-4-phenyl-3-butenyl]-2′-isobutyl-2′-(methanesulfonyl)-4-methylvalerohydrazide (Ro 327315);-; and the TNFR 1 inhibitors may comprise SODD or I-kB. It is apparent for any expert in the art that any compound or molecule may be employed in the inventive treating method with the compound or molecule reducing the TNF levels, particularly the TNF levels in the substantia nigra. The treatment of the invention also includes agents that can modify TNF levels in the brain, including not only substances acting locally but also in the periphery. Examples of these agents are: peripheral infections, inflammatory compounds such as cytokines, prostaglandins, endotoxins, etc.

It is even another object of the invention to provide a method of treating a neurodegenerative disease such as a Parkinson's disease in a mammal in need of such treatment, the method comprising: i) determining the TNF levels and detecting an inflammatory disease in a group of mammals; ii) selecting those mammals having high TNF levels and/or an inflammatory disease; and iii) administering to those selected mammals a gene therapy that reduces the TNF levels, wherein the therapy that reduces the TNF levels comprises administering an effective amount of at least one nucleic acid selected from the group consisting of nucleic acids that encode TNF ligand, TNF down-expression regulating molecules, TNF down-biological activity regulating molecules, methaloproteases inhibitors, TNFR 1 inactivating molecules and TNFR 1 inhibitors.

It is another object of the invention to provide a method of treating a neurodegenerative disease such as a Parkinson's disease in a mammal in need of such treatment, the method comprising: i) administering an effective amount of at least one TNFR 2 activating molecule or TNF signal transduction pathway molecule and an effective amount of at least one compound that reduces TNF levels, wherein the TNF signal transduction pathway molecules may be NF-KB, MAP-K, PKB/Akt, MnSOD, or GDNF, TIP-b1. The treatment with at least one TNFR 2 activating molecule or TNF signal transduction pathway molecule is neuroprotective or delays the neurodegenerative process in the substantia nigra of mammals and the treatment with at least one TNF down regulation molecule reduces the TNF production or induces low TNF expression for generating low TNF levels in the substantia nigra.

It is yet another object of the invention to provide a method of designing a treatment schedule for Parkinson's disease, the method comprising: i) determining the TNF levels and inflammatory disease in a group of patients, ii) selecting those patients having low or no TNF levels; and determining a dose of molecules that slightly increases the TNF levels, wherein said molecules are selected from the group consisting of TNF, TNF analogous, TNF active muteins, TNF up-regulating molecules and combinations thereof, or iii) selecting those patients having high TNF levels; and determining a dose of TNF down-regulating molecules, wherein the TNF down-regulating molecules are selected from the group consisting of TNF ligand, TNF down-expression regulating molecules, TNF down-activity regulating molecules, methaloproteases inhibitors, TNFR 1 inactivating molecules; TNFR 1 inhibitors and combinations thereof.

It is still another object of the invention to provide a transgenic knock in mouse as a Parkinson's disease animal model, wherein at least the somatic cells of said mouse comprise a transgene comprising a DNA sequence that encodes mTNF in operable linkage with the endogenous engrailed-1 promoter; and a loxP interference cassette downstream of the transgene, wherein said mTNF coding sequence is expressed at low levels in the substantia nigra. The transgenic knock in mouse of the invention comprises an mTNF coding sequence under the control of a CRE/loxP switch expression system, wherein the administration of CRE recombinase to the mouse develops a Parkinson's phenotype and increases the TNF levels, and mimics Parkinson's pathological conditions.

It is even another object of the present invention to provide a method of producing a transgenic knock-in mouse as a Parkinson's disease animal model, the method comprising the steps of: i) transferring a transgene into a mouse embryonic stem cells, wherein the transgene comprising a DNA sequence encoding mTNF operable linked to the endogenous engrailed-l promoter, and a loxP interference cassette downstream of the DNA sequence; ii) selecting an embryonic stem cell line that comprises said transgene, iii) transferring the embryonic stem cell of step ii) into a blastocyst, iv) transferring the blastocyst containing the transgene of step iii) into pseudopregnant foster mother, v) screening for a transgenic mouse born to the foster mother which includes the transgene, wherein said transgene of transgenic mouse born to the foster mother is expressed at low level in the substantia nigra.

It is still another object of the present invention to provide a method of testing a candidate agent for treating a Parkinson's disease, the method comprising:

i) providing a first and second transgenic knock-in mouse as a Parkinson's disease animal model, wherein at least the somatic cells of said mouse comprises a transgene comprising a DNA sequence encoding mTNF in operable linkage with the endogenous engrailed-1 promoter; and a loxP interference cassette downstream of the transgene, wherein said mTNF coding sequence is expressed at low levels in the substantia nigra;

ii) injecting to said first transgenic knock-in mouse a replication-deficient adenoviral vector, wherein said adenoviral vector expresses a nuclear form of Cre recombinase, and injecting to said second transgenic knock-in mouse a control adenoviral vector;

iii) administering to said first transgenic knock-in mouse a candidate agent; and

iv) comparing Parkinson's pathological conditions of said first transgenic knock-in mouse of step iii) to the Parkinson's disease parameters of said second transgenic knock-in mouse, wherein the reduction in Parkinson's pathological conditions in said first transgenic knock-in mouse indicates that said administrated candidate agent is potentially useful for Parkinson's diseases treatment.

It is another object of the present invention to provide a use of TNF transduction pathway molecules in the manufacture of a medicament for a Parkinson's disease treatment, wherein said TNF-related molecules are selected from the group consisting of intracellular TNF-induced molecules, extracellular TNF-induced molecules, TNF analogous, TNF active muteins, TNF active fragments and TNFR 2 activating molecules, and wherein the administration of a pharmaceutically effective amount of said medicament induces a neuroprotective effect.

It is another object of the present invention to provide a use of TNF down regulating molecules in the manufacture of a medicament for a Parkinson's disease treatment, wherein said TNF down regulating molecules are selected from the group consisting of TNF ligand, TNF down-expression regulating molecules, TNF down-biological activity regulating molecules, methaloproteases inhibitors, TNFR 1 inactivating molecules; TNFR 1 inhibitors; and wherein the administration of a pharmaceutically effective amount of said medicament reduces the TNF levels.

### BRIEF DESCRIPTION OF THE DRAWINGS

FIG. 1 shows the generation of knock-in mice of the present invention harbouring a TNF trangene under the control of the endogenous engrailed-1 promoter. a) Homologous recombination between the targeting construct and the engrailed locus. Arrows indicate primers used to detect genomic recombination. Arrowheads show primers used to detect trangenic TNF mRNA by RT-PCR. 5′IS: probe used to detect the correct insertion of the transgene in B. B) Southern blot showing correct transgene insertion in ES cells and knock-in animals. In: band corresponding to the wild-type gene locus, asterisk indicates the targeted gene locus. Wt: wild-type ES cell clone. PGK: phosphoglycerate kinase promoter, TK: thymidine kinase, Eng: engrailed, 3VI: Embryonic stem cell clone, k-in: knock-in mice.

FIG. 2 shows knock-in mice, adenoviral vectors and the CRE/loxP system of the invention to achieve different levels of TNF expression in the s.n. a) X-gal staining and immunohistochemistry for tyrosine hydroxilase (TH) of nigral sections injected with Adbetagal. Three representative sections (at positions AP:−2.8; −3.4; −3.8 from bregma according to Paxinos) and encopassing the whole subtantia nigra are shown. b) detection of nuclear CRE (left panel), TH (center panel) and colocalization of CRE and TH (right panel) in the s.n. as a analyses by double immunofluorescence. c) quantitation of double labeled TH/betagalactosidase or TH/CRE throughout the entire s.n. as percentage of double labeled cells/total TH cells 4 days after adenoviral transduction in that region. d) Detection of CRE-mediated recombination in the s. n. Genomic DNA from the s.n. of control littermates (wt) or knock-in (k-in) mice was extracted and amplified by PCR. The expected band of recombined DNA after excision of the Neo cassette is shown. GAPDH amplification was used as a control of the amount of DNA subjected to PCR. C: control PCR without the addition of DNA. ML [symbol] 100 bp ladder molecular weight. Sal.: knock-in mice injected with saline. e) RT-PCR of the s.n. or hippocampus of knock-in mice or control littermates (wt). Basal expression of transgenic TNF could be observed in the s.n. of untreated and Adbetagal-injected knock-in mice. TNF is upregulated in knock-in animals injected with AdCRE at 7 and 20 days. Amplification of GAPDH RNA retrotranscribed and amplified as the rest of the samples was used as a control of the amount of RNA subjected to PCR. Hip: hippocampus. f) immunohistochemistry for TNF. Representative sections of the s. n. of knock-in mice after 20 days of being injected with AdCRE (left panel) or Adbetagal (right panel) are shown. A higher magnification of a neuron expressing TNF is shown in an inset. Scale bars, panel a and f=200 um; panel b=20 um.

FIG. 3 is shows the progressive degeneration of TH+ neurons in the s.n. of knock-in mice after AdCRE injection in this brain region. Representative sections at three different levels of the s.n. was immunostained against tyrosine hydroxilase 20 (a, b, c) and 100 (e, f, g) days after adenoviral administration. a) and e) s.n. contralateral to AdCRE injection, b and f) s.n. ipsilateral to AdCRE injection, c and g) s.n. ipsilateral to Adbetagal injection. d) quantitation of the % of TH+ neurons in the whole s.n. comparing the ipsi vs contralateral side 20 days after treatment. $p<0.001 (knock-in mice injected with AdCRE vs. similar animals injected with Adbetagal), *p<0.0001 (non-injected compared with all injected animals except knock-ins with AdCRE), &p<0.001 (knock-in mice injected with Adbetagal vs. control littermates injected with either vector). g) quantitation of the % of TH+ neurons in the whole s.n. comparing the ipsi vs the contralateral side 100 days after treatment. ) $p<0.0001 (knock-in mice injected with AdCRE vs. similar animals injected with Adbetagal), and *p<0.05 (non-injected compared with all injected animals except knock-ins with AdCRE), (Tuckey unequal N Post Hoc test). Numbers in the figures denotes mm from bregma, according to Watson and Paxinos. Values are median percentage of neurons±s.e.m. k-in: knock-in animals. wt: control littermates. Ad: animals injected with AdCRE or Adbetagal

FIG. 4 shows the progressive induction of rotational behavior in knock-in mice of the invention injected with AdCRE after apomorphine administration. (a) Number of contralateral turns at 20 (grey), 40 (stripped), 60 (white) and 80 (black bars) days post adenoviral injections in the s. n. Values from responders and non-responder animals were separated for graphication. b) Correlation between net contralateral turns and amount of TH in the striatum of individual knock-in animals injected with AdCRE. The correlation has an R²=99.238

FIG. 5 shows the constitutive levels of TNF protects dopaminergic neurons against 6-OHDA toxicity (a-e) Immunostaining against TH in the s. n. ipsilateral to adenoviral vector injections, 20 days after 6-OHDA inoculation in the striatum. a) knock-in mice injected with saline, instead of 6OH-DA. b-e) 6OH-DA injected animals. b) control littermates treated with saline c) knock-ins treated with saline d) knock-ins treated with Adbetagal, e) knock-ins treated with AdCRE, f) Quantitative analysis of the remaining TH+ neurons, ipsi vs contralateral. *p<0.0002 (knock-in mice injected with saline vs. any of the other groups), # p<0.0002 (6OH-DA treated animals: knock-in mice with no adenoviral treatment, but injected in the s.n. with saline vs control littermate similarly handled), $p<0.001 (knock-in mice injected with 6OH-DA and treated with saline vs. knock-in mice injected with 6OH-DA and treated with Adbetagal), &p<0.05 (knock-in with AdCRE vs. knock-in with Adbetagal). g) net turns after apomorphine injection of similar groups of animals. *p<0.0001 (saline-injected knock-in animals vs all 6OH-DA injected mice)

### DETAILED DESCRIPTION OF THE INVENTION

Unless defined otherwise, all technical and scientific terms used herein have the same meaning as commonly understood to one of ordinary skill in the art to which this invention belongs.

The term “transgene” is used herein to describe genetic material which has been or is about to be artificially inserted into the genome of a mammal, particularly a mammalian cell of a living animal.

By “Parkinson's disease” (“PD”) is meant a condition associated with [resting tremor, rigidity, bradykinesia, gait abnormalities and postural inestability]

“Transgenic animal” means a non-human animal, usually a mammal (e.g., mouse, rat, rabbit, hamster, etc.), having a transgene present as an extrachromosomal element in a parts of its cells or stably integrated into its germ line DNA. The trangene is introduced into the germ line of such transgenic animals by genetic manipulation of, for example, embryos or embryonic stem cells of the host animal.

A “knock-in mouse” means an alteration in a mouse cell genome that results in altered expression (e.g., decreased (including ectopic)) of the target gene, e.g., by introduction of an additional copy of the target gene. “Knock-in transgenic mouse” of interest for the present invention can be transgenic animals having a knock-in of the animal's endogenous TNF under the control of site-specific promoter, for example the engarailed-1 promoter driving the expression of TNF in the substantia nigra.

The term “switch expression” as used herein refers to an expression system wherein a specific gene can be expressed at a desired time. For example, the switch expression may be constructed by introducing a sequence, which can be removed at a desired time, downstream a gene to be expressed. An example is Cre/loxP expression system. The “Cre/loxP expression system” comprises an insert gene that has been interposed between two loxP sequences, which is downstream to the gene of interest so as to suppress expression of the gene; and a P1 phage Cre DNA recombinase enzyme (“Cre”) which removes the insert gene together with one of the loxP sequences. The gene of interest can be expressed at any time by action of Cre.

The term “transcriptional regulatory sequence” as use herein refers of an initiation signals, enhancers, and promoters, which induce or control transcription of protein coding sequences with which they are operably linked. In preferred embodiments, transcription of the TNF gene is under the control of a promoter sequence (or other transcriptional regulatory sequence) which controls the expression of the recombinant gene in a cell-type in which expression is intended.

In clarifying an onset mechanism of a human disease and developing means for treating the disease, an animal model presenting pathological conditions very similar to those of the disease plays an important role. However, as described above, a Parkinson's disease transgenic animal model has not yet been produced. An object of the present invention is to provide a novel transgenic animal model that exhibits the pathological conditions analogous to human Parkinson's disease. The transgenic knock-in mouse of the invention is also useful for studying the effects of different TNF levels on the s.n.

It is to be understood that this invention (transgenic animals and uses thereof) is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention.

According to one inventive example of the Parkinson's disease transgenic animal model, cDNA derived from TNF is introduced into the animal such that the cDNA is switch-expressed. The switch expression is for example, a CRE/loxP switch expression system. By switch expression of the TNF-derived cDNA, a TNF can be produced at a high level giving, for example, a mouse with mimic Parkinson's disease.

The Parkinson's disease transgenic animal model of the present invention exhibits pathological conditions analogous to the Parkinson's disease. The pathological conditions include, while not limited thereto, progressive death of dopaminergic neurons in the substantia nigra pars compacta, progressive dissapearence of tyrosine hydroxylase in the striatum, rotational behaviour after apomorphine administration.

These pathological conditions in the human beings include, while not limited thereto, progressive death of dopaminergic neurons in the substantia nigra pars compacta, progressive dissapearence of tyrosine hydroxylase in the striatum, asymmetry of motor symptoms.

The method for producing an animal model according to the present invention is described below.

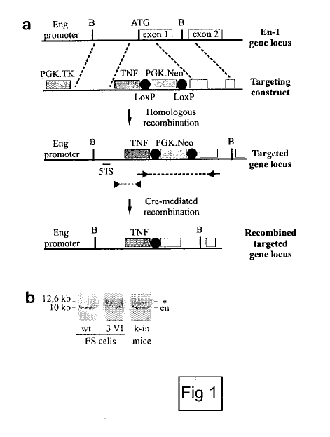

The inventors have been constructed knock-in mice in which the expression of TNF is under the control of the endogenous engrailed-1 promoter (FIG. 1a). Generation of knock-in transgenic mouse of the invention was made by homologous recombination between the targeting construct and the engrailed locus. According to a preferred embodiment the targeting construct comprises the TK gene under the control of phosphoglycerato kinase promoter (PGK), a nucleic acid sequence homologous to autologous engrailed 5′non-coding sequence, TNF gene, loxP sequence, Neomocine gene under the control of PGK promoter, loxP sequence and a second nucleic acid sequence homologous to autologous engrailed gene. After the homologous recombination the TNF gene is under the control of the site-specific autologous engrailed promoter. This promoter directs the expression of the transgene (TNF gene) to substantia nigra region (FIG. 1a). Targeting construct may comprise transgenes other than TNF gene. This other transgenes may be any transgene the over-expression of which in the s.n. is to be studied.

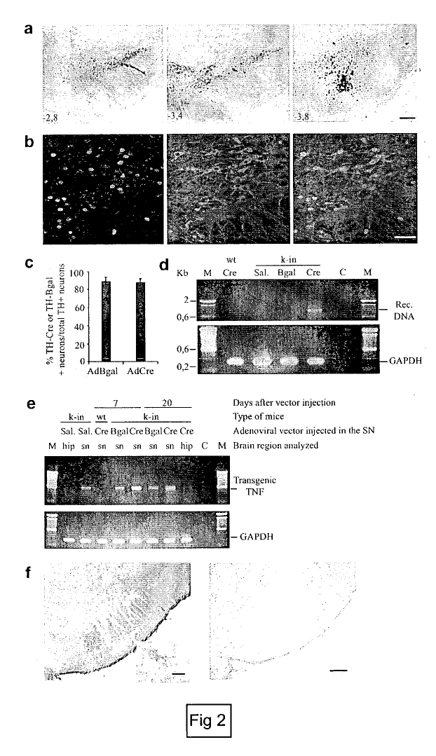

The targeted insertion of the construct after homologous recombination could be observed by Southern Blot analysis in ES cells and adult mice (FIG. 1b). By RT-PCR, a specific band for transgenic TNF could be observed in the s.n. but not in other brain regions using primers that recognize TNF and engrailed 5′non-coding sequences in naive animals (FIG. 2e, lanes knock-in injected with saline instead of adenoviral vectors (Sal)). However, the expression of the transgene in the animals could not be detected by ELISA, immunohistochemistry or in situ hybridization with specific antibodies and probes (data not shown).

The Neo cassette (with its phosphoglycerate kinase promoter) can interfere with the expression of the transgene. This strategy has been used to generate several animal models of disease where the transgene was expressed at different levels in so-called hypomorphic mice (Lewandoski, M. Nat Rev Genet 2: 743-55, 2001, herein cited as reference). Since the neo cassette was floxed. The transgenic knock-in mice of the present invention had low TNF expression that could be upregulated by the elimination of this cassette by CRE-mediated recombination in the s.n. The over-expression of a TNF gene can be made by any known method, for example by creating a targeting knock in construct that comprises the TNF gene under the control of transcriptional regulatory sequence that increases the transcription of such TNF gene. The objective is to create a knock-in mouse that expresses high levels of TNF in s.n. as a Parkinson's disease model. Hence, any transgenic mice expressing high TNF levels in the s. n. falls within the scope of the present invention. Alternatively, a ubiquitous promoter could be used and TNF could be up-regulated in the brain or body region of interest via the administration of the adenoviral vector expressing Cre in the region of interest. The promoters include, while no restricted thereto, a c-kit promoter: specific for CA1, CA2 and CA3 regions of the hippocampus, the anterior region of the dentate gyrus, and to the ganglion cell layer of the retina; calcium/calmodulin kinase II alpha promoter that drive the expression of high levels in hippocampus, cortex, and amygdala, and lower levels were detected in striatum, thalamus, and hypothalamus; NMDA-type glutamate receptor GluRepsilon3 subunit gene that drive the expression to Cerebellar granule cell-specific.

To achieve CRE expression in the s.n. we generated a replication-deficient adenoviral vector (AdCRE) expressing a nuclear form of CRE recombinase under the control of the cytomegalovirus promoter. The CRE-mediated recombination permits high TNF expression. Even in another preferred embodiment AdCRE is injected into transgenic mice of the invention in an amount enough to facilitate the expression of part or all recombined cells with the targeting construction. The possibility of regulating the amount of cells of the s.n. that expresses TNF permits to use the transgenic mice model of the invention for generating mice with different levels of TNF, the mice being useful for studying the effects of the several TNF levels of the s.n. in the Parkinson's disease.

In a embodiment, one single injection of 1×10⁸ particles of AdCRE or adenovectors expressing bacterial betagalactosidase (Adbetagal), could transduce 89.6±4.1 and 88.0±4.0% of tyrosine hydroxilase (TH+) positive cells all over the s.n., respectively (FIGS. 2a, b and c, n=3/group). Most of AdCRE transduced cells were TH+ neurons (FIG. 2b). Transduced cells outside the s.n. could be observed and account for 10% of all transduced cells, transgene expression lasted for at least 100 days and signs of inflammation could be observed at day 7 post-injection that resolved by day 20 after administering any of the two vectors.

The expected CRE-mediated deletion of the floxed Neo cassette could be observed at 7 days in the s.n. of knock-in mice of the invention but not in control littermates transduced with AdCRE and also not in knock-in mice of the invention injected with Adbetagal or saline (FIG. 2d). DNA recombination in the mice of the invention was paralleled by an upregulation of TNF expression in the s.n. both at 7 and 20 days after adenoviral injection (FIG. 2e). This result was confirmed by immunohistochemistry against TNF (FIG. 2f). No TNF overexpresion was observed in a control region (the hippocampus) or in knock-in animals injected with Adbetagal as seen by RT-PCR and immunohistochemistry (FIGS. 2e and f). These results show that the mice generated were hypomorphic and could be used to study the effects of the chronic expression of different TNF levels on the viability of dopaminergic neurons in the s.n. Moreover, targeted and efficient delivery of CRE recombinase into the s. n. of mice using adenoviral vectors upregulates TNF expression specifically in this region. Moreover, targeted and regulated delivery of CRE recombinase into s.n. region using such adenoviral vector upregulates TNF expression at different levels.

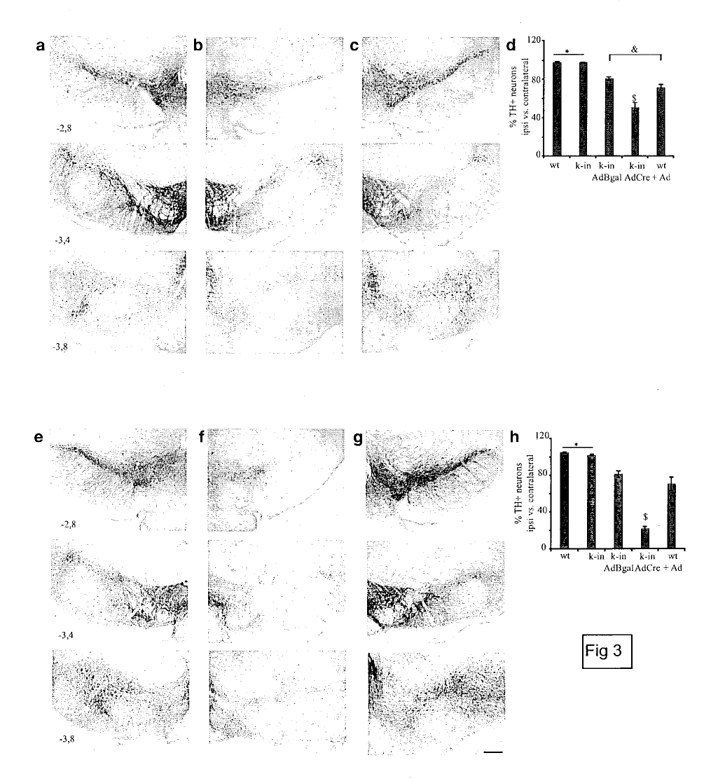

In order to assess the potential effect of the long-term upregulation of TNF on neuronal viability, AdCRE and Adbetagal were injected (n=4/group) in the s.n. of transgenic mice of the invention and control littermates. Uninjected animals (n=2/group) were used as controls for the adenoviral injection. Tyrosine hydroxilase positive (TH+) cells were identified by immunohistochemistry and counted throughout the s.n at 20 and 100 days. A statistically-significant 30% decrease in the number of TH+ cells was observed following AdCRE injection into knock-in mice of the invention compared with Adbetagal-injected knock-in mice of the present invention 20 days after treatment (FIG. 3d $p<0.001 and compare FIGS. 3b and c). At 100 days after AdCRE administration, only 20% of TH+ cells remained detectable in the knock-in animals (FIG. 3h, $p<0.0001 and compare FIGS. 3f and g). The observed effect of TNF was not due to a downregulation of TH expression but to a true loss of neuronal bodies as assessed by Niss1 staining (data not shown). This effect was also not mediated by a toxic effect: of CRE expression itself since CRE expression caused no reduction in the number of neurons in the s.n. in control littermates (FIGS. 3f and g, wt+Ad). The injection of adenoviral vectors per se resulted in a loss of 20-30% of TH+ cells at 20 and 100 days (compare injected and uninjected animals FIGS. 3d and h, *p<0.05 to 0.0001). This effect was observed mainly only on the site of injection but not in the anterior or posterior s.n. (FIGS. 3b, c, f and g). No effect of any adenoviral injection could be observed on the contralateral site 20 or 100 days after treatment (FIGS. 3a and e and data not shown).

When knock-in mice of the present invention (expressing TNF constitutively in the s.n.) injected with Adbetagal were compared to control littermates (not expressing TNF) injected with Adbetagal or AdCRE, a statistically significant increased number of TH+ neurons could be detected in the first group at 20 but not at 100 days (FIGS. 3d and h, knock-in Adbetagal vs wt Ad, (&p<0.001 at day 20 and p=0.205 at day 100). This result showed a first indication of a transient protective effect of the TNF expressed constitutively on the loss of neurons derived from the unspecific effect of the adenoviral injection. Additionally, long-term expression of upregulated TNF in the s. nigra is neurodegenerative.

The possible mechanisms of action of TNF in mediating neuronal death in the s.n. may be a number of mechanisms as stated above. The mechanism of TNF action in neurodegeneration could be, our data provide evidence that TNF can trigger neuronal death in the s.n. Thus, a treatment for decreasing TNF levels in the s.n. provides a useful tool for treating patients with Parkinson's disease. According to a embodiments of the invention the treatment could consist of administrating TNF ligand, TNF down-expression regulating molecules, TNF down-biological activity regulating molecules, methaloproteases inhibitors or other inhibitors for inhibiting the TNF in the patients. For example, those TNF inhibitors disclosed in publication WO 00/18409, herein cited as reference.

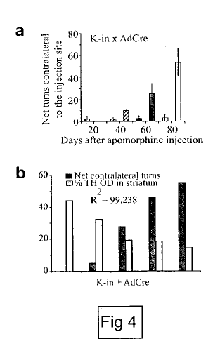

In order to demonstrated that the decrease in TH+ neurons in the s.n. observed in AdCRE-injected knock-in mice of the present invention had any functional consequence on the motor behaviour of these animals. Knock-in mice and littermate controls were injected with AdCRE and control adenoviral vectors (Adbetagal) and after 20, 40, 60 and 80 days apomorphine was administered and rotational analysis of the animals was performed. For this experiment, 16 knock-in animals were injected with AdCRE, and all the other groups have 6 animals each. Only some knock-in animals injected with AdCRE showed a significant increment in the net contralateral turn after apomorphine injection, starting 40 days after adenoviral injection (FIG. 4a). This apomorphine-induced behaviour augmented with time (FIG. 4a). A closer evaluation of the data revealed the existence of two different groups among these animals. At 40 days after AdCRE inoculation, 46% of the treated animals were showing an increment in their net turn after apomorphine administration while the rest of the same group did not show this behaviour (FIG. 4a and data not shown). At 60 days, a slightly higher percentage (54%) of the animals showed net contralateral turns after apomorphine and this persists till the last time point analysed (80 days) (FIG. 4a and data not shown).

This difference in the rotational behaviour of the same group of animals could be due to a difference in the number of TH+ cells left in the s.n. or the amount of TH density in the striatum after AdCRE treatment. An inverse, exponential and highly significative correlation was observed between the amount of TH optical density quantified in the striatum at 100 days and the number of net turns of the animal after apomorphine treatment (FIG. 4b, p<0.0005, n=5). Among the animals of this group, no significant differences could be observed in the number of TH+ neurons in the s.n. at 100 days.

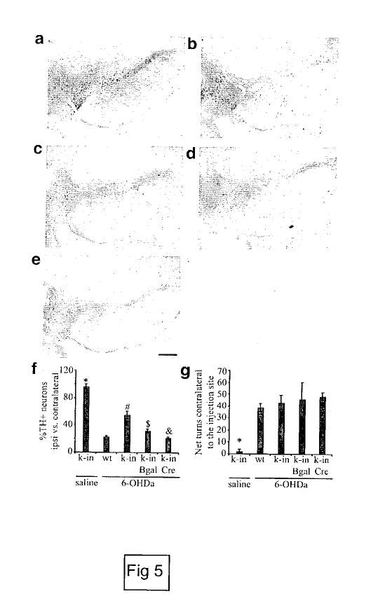

In addition, the number of TH+ cells in the s.n. left in the knock-in mice remains fairly constant 100 days after AdCRE injection. A drawback of this system to study dopaminergic cell death is that the adenoviral injection per se seems to cause the death of ca. 20% of neurons in the s.n. at the site of injection (FIG. 3). Nevertheless, this effect did not obscure the present results since minor differences in cell viability could be still statistically determined (FIGS. 3 and 5). The action of TNF seems to add-up to the neurodegenerative effect of the 6OH-DA (see FIG. 5). Both molecules certainly convey their signals through different pathways, but they might have or have not a final effector molecule. In these experiments, only 54% of the animals showed a rotational behaviour when apomorphine was administered to them 60 to 80 days after adenoviral inoculation. One way to obtain a homogenous population of animals lesioned in the striatum, could be to pre-test animals with apomorphine before analysis: only those with more than 75% loss of TH optical density in the striatum will show a rotational behaviour (FIG. 4).We could be conclude that long-term expression of upregulated TNF in the s.n. affected the behaviour.

According to the invention, transgenic mouse models useful for screening active drugs are provided. The animals have up-regulated the expression of TNF. The transgenic mouse model of the invention is useful for testing the specificity of drugs for Parkinson's disease treatment. In addition, these animals provide a useful model for the behavioral testing of certain candidate active compounds and contribute to the efficacy of drugs in current use for Parkinson's disease.

By upregulating the TNF levels, we have generated a new transgenic knockin mouse as a model of progressive neurodegeneration in the s.n. The model includes such transgenic knock-in mouse plus CRE/loxP switch expression system. This new transgenic knock-in mouse model is useful for neurodegenerative diseases studies, preferentially Parkinson's diseases.

The rate of death of dopaminergic neurons/time produced by our combination of techniques is -slower -than the one described for other common used models, e.g. MPTP or 6OH-DA administration. . In the first case, the neurodegeneration observed at 10 days after treatment comprises around 50% of the dopaminergic neurons in the substantia nigra. In the second case, the injection of 6OH-DA should cause the death of 20 and 50% of the dopaminergic neurons after 7 and 14 days, respectively. In our model, 50% and 80% of neuronal death is achieved 20 and 100 days post-AdCRE inoculation. Therefore, the inventive model permits to obtain, after transduction with adenoviral vector expressing CRE, a variety of animals with several degrees of the TNF gene expression (hypomorphic mice). The inventive transgenic mouse model differeing from the known models (see Sauer, H., et. al.,. Neuroscience 59:401-415, 1994; Beal, M. F.. Nat Rev Neurosci 2:325-334, 2001; and Betarbet, R., et. al.,. Nat Neurosci 3:1301-1306, 2000, herein cited as reference) permits to better simulate the development of a Parkinson's disease in the human beings. The inventive transgenic mouse model develops the disease more slowly as compared to other known models.

We have been shown that pathological conditions of the transgenic knock-in mice of the invention are very similar to those corresponding to a human Parkinson's disease. The pathological conditions could be developed in a mouse by introducing TNF-derived cDNA into the mouse such that the cDNA was switch-expressed, whereby the present invention was accomplished. Thus, the present invention is a Parkinson's disease transgenic animal model into which cDNA derived from TNF gene has been introduced.

The combination of the CRE/loxP system, adenoviral vectors and the endogenous engrailed promoter of the invention in knock-in mice with a hypomorphic allele allowed the control transgene expression specifically in the s.n. of adult mice. This is the first example in which the basal expression of a transgene driven by an endogenous promoter is upregulated by the delivery of an adenoviral vector expressing CRE in the s.n. By placing the loxP sites correctly, these combination of techniques should provide valuable for the regional and temporal expression (or suppression of expression) of genes of interest in the s.n. of adult mice.

The TNF expressed constitutively in the knock-in mice of the invention effect on neuronal survival (FIGS. 3d and h, knock-in+Adbetagal vs wild type+Ad). To investigate the effect of the constitutive, low expression of TNF in the s.n. of these animals, we denervated the striatum by injecting 6OH-dopamine in this region and the effect of this treatment on TH+ neurons in the whole s.n., TH optical density in the striatum and motor behaviour after apomorphine injection were evaluated (n=4 per group). A significant, 3 fold increase in the number of TH+ neurons could be observed in 6OH-DA-treated knock-in mice compared to control littermates at 21 days after 6OH-DA administration (FIG. 5f and compare FIG. 5c with 5b , #p<0.0002). This neuroprotective effect of the constitutively expressed TNF was not enough to reflect differences in the rotational behaviour of the animals or the striatal density of TH (FIG. 5g and data not shown).

Knock-in mice of the invention were injected with AdCRE or Adbetagal 7 days before 6OH-DA administration and the effects of the adenoviral inoculation were analysed. The adenoviral vector per se had a negative effect on the survival of the neurons in the s.n. (FIG. 5f and compare FIG. 5c, with d, $p<0.001). Interestingly, there was a significant decrease in the number of TH+ cells in the s.n. of AdCRE-injected knock-in animals when compared to the Adbetagal mice, showing again the neurodegenerative effect of the upregulated TNF on dopaminergic neuronal survival, visible even after treatment with 6OH-DA (FIG. 5f and compare FIG. 5d and e, &p<0.05).

We show that low levels of TNF could be neuroprotective against the toxicity of 6OH-dopamine. This protective effect was only seen on neuronal cell bodies but not on their terminals in the striatum as seen by TH density in this last region. The lack of protection of the striatal terminals was correlated with no difference in the turning behaviour of any of the experimental groups of animals (FIG. 5). A similar phenomenon has been reported for glial cell line-derived neurotrophic factor (GDNF), the most potent neuroprotective molecule for dopaminergic neurons described to date (Choi-Lundberg, D. L. et al. Science 275: 838-841, 1997; Bilang-Bleuel, A. et al, Proc Natl Acad Sci USA 94: 8818-8823, 1997 and Kordower, J. H. et al. Science 290: 767-773, 2000, herein cited as reference). Thus, long term expression of low levels of TNF in the s.n. is neuroprotective.

The putative neuroprotective mechanisms of TNF could also be multiple. Binding of TNF to its type I receptor not only activates an apoptotic pathway but also induces NF-kB translocation to the nucleus, acting in the majority of cases studied as a survival signal for neurons (Mattson, M. P., el. al; J Neurosci Res 49: 681-697, 1997 and Kaltschmidt, B., et. al; Proc Natl Acad Sci USA 96: 9409-9414, 1999, herein cited as references). Interestingly, binding of TNF to its type II receptor activates mainly the NF-kB pathway but not the apoptotic one (Mukhopadhyay, A., et. al. J Biol Chem 276: 31906-31912, 2001, herein cited as reference). TNF in its membrane form could be acting via type II receptor and mediate neuroprotection perhaps via activation of PKB/Akt (Fontaine, V. et al. J. Neurosci. 22: 216, 2002, herein cited as reference). In addition, TNF has been shown to induce the enzyme MnSOD that could scavenge the free radicals produced by the dopaminergic toxin 6OH-DA (Rogers, R. J., et. al., J Biol Chem 276. 20419-20427, 2001, herein cited as reference). Finally, since TNF was shown to induce GDNF, and-this neurotrophic factor also protects neuronal bodies but not their terminals when adminsitered in the s.n., it could be possible that TNF exerts its neuroprotective effect via GDNF (Appel, E., et. al., Neuroreport 8: 3309-3312, 1997, herein cited as reference). Therefore, with the combination of the above disclosed neuroprotective effect with the neuroprotective mechanisms of TNF also above disclosed, the inventive method involves the administration of TNF signal transduction pathway compounds as intracellular TNF-induced molecules, extracellular TNF-induced molecules, TNF analogous, TNF active muteins, TNF active fragments, or TNFR 2 activating molecules for treating a Parkinson's disease, preferably when the patient has low TNF levels or no TNF. The TNF signal transduction pathway compounds comprise, while not restricted thereto, NF-kB, MAP-K, PKB/Akt MnSOD, GDNF and/or TIP-b1 molecules

Up-regulated levels of TNF resulted in a progressive loss of DA neurons in the s.n. but constitutive expression of TNF showed to be, unexpectedly, neuroprotective. In the light of our results, a careful examination of the levels of TNF in PD patients and experiments involving different TNF levels in already-lesioned animals will be required before designing a therapy that directly or indirectly modifies TNF levels. Our results on neurons of the s.n. suggest that the levels of cytokine studied should be considered before drawing conclusions on the role of cytokines and, specifically of TNF, on neuronal survival.

As shown above, low concentration of TNF provides a neuroprotective effect and high concentration of TNF provides a neurodegenerative effect in s.n. According to the invention, a treatment with TNF is provided wherein the concentration of TNF in the patients affected by the Parkinson's disease is regulated. In addition, a treatment in which molecules activate a survival signal into neurons via TNF type II receptors is also provided. More specifically, the inventive method is for treating a patient affected by Parkinson's disease. Any agent capable of modifying the TNF expression may be used, such as TNFR 2 activating molecules, extra-cellular TNF induced molecules (for example, GNDF, MnSOD and TIP-b1), intra-cellular TNF induced molecules (for example, NF-KB, MAP-K, PKB/Akt) or TNF down-regulating molecules, as stated above.

Specifically, in a preferred embodiment the invention provides methods for treating Parkinson's disease by reducing production or expression of TNF; or by regulating production or expression of TNF at low levels as a neuroprotective mechanism.

All mentioned publications are herein incorporated by reference for the purpose of describing and disclosing, for example, the transgenic animals, TNF regulating molecules, constructs, systems and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

It should be understood that the foregoing relates to preferred embodiments of the present invention and that numerous changes may be made therein without departing from the scope of the invention. The invention is further illustrated by the following examples, which are not be construed in any way as imposing limitations upon the scope thereof. On the contrary, it is to be clearly understood that resort may be had to various other embodiments, modifications, and equivalents thereof, which, after reading the description herein, may -suggest themselves to those skilled in the art without departing from the spirit of the present invention and/or scope of the appended claims.

### EXAMPLES

### Example 1

### Knock-in Mouse Construction

The 5′ homologous sequences of the engrailed-1 gene from pPro-en1/5 were cut out using Xba I and Asp718 I and were filled with Klenow polymerase and cloned in front of a TNF cDNA cassette and generated the 8.1 kb pAS7 plasmid. The plasmid pBluescript SKII was cut with Not I and Asp718 I and the original multiple linker sequence was replaced by a synthetic linker sequence with Not I-SgfI-XbaI EcoRV-SgfI-Asp718 and was designated pAS10. The En-1 5′ homologous region with the cDNA cassette was inserted into pAS10 by cutting both pAS10 and pAS7 with XbaI and EcoRV and ligation of the insert from pAS7, thus generating pAS11. By linker addition into the construct pKSloxPNT into the EcoRI site an additional SgfI site was generated (pAS9). Removal of the 5.2 insert from pAS11 was done by cutting with PvuI. The fragment was then cloned into the SgfI site of pAS9 generating pAS13. pAS13 was linearized by cutting with Asp718 I (Roche Diagnostics). 30-60 μg linearized plasmid was used for electroporation of 1.5×10⁷ embryonic stem cells using a gene pulser (Biorad) at 25 μF, 0.4 kV, 400 Q, 0.9 msec. ES cells were grown on neo-resistant feeder layers and selected with G418 and ganciclovir until cell clones were visible. Positive clones were tested by southern blotting and injected into CD1 (Swiss) blastocysts and transferred into anesthesized pseudopregnant foster mothers. Highly chimeric animals were used to generate founder animals. All animal procedures were performed according to the rules and standards of the german animal protection law.

### Example 2

### Adenoviral Construct

AdBgal was gently provided by Dr. Mallet, (Hospital Pitie Salpetriere, Paris) and has been previously described (Le Gal La Salle, G. et al.,, Science 259:988990, 1993, herein cited as reference). The recombinant adenoviral vector expressing Cre recombinase (AdCre) was constructed following standard procedures described in detail in Revah, F. et al., Gene transfer into the central and peripheral nervous system using adenoviral vectors, Chapter 7, Ed. John Wiley and Sons, 1996, herein cited as reference. Briefly, the cDNA for Cre was excised from the pBS185 plasmid (GibcoBRL, Gaithersburg, Md.). A nuclear localization signal (NLS) was added to target the protein to the nucleus by PCR as in Kanegae, Y. et al,. Nucleic Acids Research 23: 3816-3821, 1995 and the new construct was sequenced. Then it was cloned in the pADPSY plasmid, under the control of the citomegalovirus promoter (CMV) and the simian virus 40 (SV40) polyadenylation signal, downstream of the adenoviral pIX gene, and upstream of the encapsidation signal. This plasmid was co-transfected with the left arm of a ClaI-digetsed and purified Ad5 into HEK293 cells (ATCC, Manassas, Va.). After signs of lysis were observed, viruses were collected from the supernatant and DNA was obtained by the HIRT procedure. Correct recombination was verified with restriction nucleases (BglII and HindIII, Gibco), and further confirmed by southern blots. Cre protein was detected by western blot from HeLa cells transduced with the AdCRE of transduced cell lysates, as a band of 32 Kda . Adenoviral vectors were agar-plaque purified. Stocks were obtained from large-scale preparations in HEK293 cells by double cesium chloride gradients, and then were plaque agar quantified (Final titers:AdBgal=1.5×10¹⁰ pfu/ul, AdCre=1×10¹⁰ pfu/ul). Endotoxin analysis was performed using E-TOXATE® Reagents (Sigma, St. Louis, Mo.), and revealed there stocks had less than 1 ng/ml of endotoxin. PCR analysis to detect E1A gene and transduction of non transcomplementary cells (HeLa, ATCC) determined that viral stocks were free of auto-replicative particles (Lochmuller, H. et al. Human Gene Therapy 5: 1485-1491, 1994, herein cited as reference).

### Example 3

### Neurodegenerative and Neuroprotective Assays in Transgenic Knock-in Mouse Model

Animal surgery:

10 and 12 week-old animals were used. They were anesthetized with intraperitoneally injected ketamine (75 mg per kg, Parke-Davis) and Rompun (500 μl per kg of a 2% solution, Bayer). Stereotaxic injections were performed with very thin glass micropipettes (Drummond Scientific Company, Broomall, Pa.), outer diameter 50-70 μm as in Bell, M. D. et al. Eur J Neurosci 8: 1803-1811, 1996.. All injections were performed in 2 μl, at 0.5 ul/min, and the capillar was left in place for 10 min before slowly retracted. Adenoviral vectors were injected at 1×10e8 pfu/μl in PBS-10% glicerol. For 6-hydroxydopamine (ICN Biomedicals Inc, Aurora, Ohio), each mice was injected with 4 ug of drug diluted in PBS-0.02% ascorbic acid. Striatal coordinates were 0.4 mm anterior, 1.8 mm right, 3.5 mm ventral, and for the SN were 3.4 mm posterior, 1.2 mm lateral, 4.5 mm ventral.

Tissue processing and immunocytochemistry:

Animals were deeply anesthetized with ketamine (500 mg per kg) and perfused through the aorta with PBS and 4% PFA (Sigma). Brains were removed, postfixed for 4 hours at 4° and cryoprotected in 20% sucrose. They were frozen in dry ice cold iso-pentane (J. T. Baker, Phillipsburg, N.J.). Serial 30 μm coronal cryosections were obtained through the SN and striatum. Free-floating sections were permeabilized with 0.5% Triton X-100. For Tyrosine Hydroxylase staining, a rabbit polyclonal antibody was used (Chemicon, Temecula, Calif.; 1:500). To detect Cre, a monoclonal antibody was used (Chemicon; 1:1000). For TNF immunocytochemistry, a rabbit polyclonal antibody was used (ENDOGEN, Woburn, Md.). Secondary antibodies were from Jackson (West Grove, Pa., 1:250). They included biotin donkey-anti rabbit, Cy2 conjugated donkey anti-rabbit and Cy3 conjugated donkey antimouse. For TH immunocytochemistry, the avidin-biotin complex method was used (ABC, Vector Laboratories, Burlingame, Calif.). For TNF, the DAKO® Catalyzed Signal Amplification (CSA) System was used (DAKO, Carpinteria, Calif.). We used 3,3′-diaminobenzidine (DAB) to visualize the final products. All antibodies were incubated in PBS+1% normal donkey serum (Sigma). Xgal staining was performed as described in Revah, F. et al Gene transfer into the central and peripheral nervous system using adenoviral vectors, Chapter 7 Ed. John Wiley and Sons, 1996. DAB and Xgal stained sections were analyzed under bright-field microscopy (Olympus BX60, Japan), images were captured with a digital camera (Cool Snap-Pro cf color, Media Cybernetics, Silver Spring, Md.) linked to an image analysis system (Image ProPlus, Media Cybernetics). Double labeled immunofluorescence images were analyzed with a laser confocal microscopy (Carl Zeiss, Germany).

PCR and RT-PCR:

Genomic DNA was obtained from s.n. samples following standard methods (Ausubel, F. K. et al. Current protocols in Molecular Biology, Ed. John Wiley and Sons, Inc., 1995). Primers used to detect cre-mediated recombination were: sense (TNFs) 5′CAT ATg CAC CAC CAT CAA gg 3′, and antisense (eng3′B) 5′CAT CTg gAg CAC ACA AgA gC 3′. These primers should amplify 970 bp when Neo is excised, and under the following PCR conditions, the expected band of 2970 bp for Neo is not observed. The PCR protocol was: 94°, 4 min; (94°, 30 sec; 55°, 30 sec; 72°, 1 min 30 sec; 35 cycles); 72°, 3 min. RNA from specific brain regions was purified with TRIzol reagent following the manufacturer's specifications (GibcoBRL). Total RNA (5 ug) was reverse-transcribed with SuperScript II RNase H+ Reverse Transcriptase (Gibco) for first-strand cDNA synthesis. PCR reaction conditions to detect the expected 570 bp band for the transgenic TNF was the same as before, but with 38 cycles. Primer sequences were: sense (eng5′B) 5′ gAg ATT TgC TCC ACC AgA gC 3′; antisense (TNFas): 5′ ggg AgT AgA CAA ggT ACA AC 3′. Amplification of GAPDH was performed following similar conditions using the following primers: sense: 5′ TGATGACATCAAGAAGGTGGTGAAG 3′, antisense: 5′TCCTTGGAGGCCATGTAGGCCAT 3′. All products were separated by gel electrophoresis and visualized by etbidium bromide staining.

Rotational analysis.

Apomorphine (Sigma) diluted in sterile water was injected to mice subcutaneously (0.25 mg per kg). They were placed in 20 cm plastic hemispherical bowls, and after a 5 min habituation period, turns ipsi and contralateral were counted for 20 min. Results were expressed as net turns contralateral to the brain injection site.

Cell counting:

The number of TH+ neurons was assessed by counting only the neurons of the s.n. pars compacta every sixth 30 μm sections along the whole s.n. (six to seven cuts per mice). The percentage of neurons remaining in the ipsilateral side, compared to the intact contralateral side, was obtained. Values were expressed as the median of this percentage ±s.e.m. of all the cuts from all the animals of each group. Statistical analysis was performed by ANOVA, followed by Tukey or Unequal N Post Hoc Tests, where appropiate. Probability values of less than 0.05 were considered statistically significant.

What is claimed is:

1. A method of treating a neurodegenerative disease in a mammal in need of such treatment, said method comprising:
i) administering an effective amount of at least one TNF-related molecule selected from the group consisting of intracellular TNF-induced molecules, extracellular TNF-induced molecules, TNF analogous, TNF active muteins, TNF active fragments, TNFR 2 activating molecules and mixtures thereof.

2. The method of claim 1, wherein the intracellular TNF-induced molecules are selected from the group consisting of NF-KB, MAP-K, PKB/Akt, JNK, p38, erks, ask1, gck, IKK, acidic and basic Sphingomyelinases, FAN, caspases and a combination thereof.

3. The method of claim 1, wherein the extracellular TNF-induced molecules are selected from the group consisting of MnSOD, GDNF, TIP-b1, BDNF, IL-6, HLA, adhesion molecules and a combination thereof.

4. The method of claim 1, wherein the neurodegenerative disease is Parkinson's disease.

5. The method of claim 1, wherein the treatment provides a neuroprotective effect or a delaying effect on the neurodegenerative process in the substantia nigra.

6. The method of claim 1, wherein the mammal is a human.

7. A method of treating a neurodegenerative disease in a mammal in need of such treatment, said method comprising:
i) administering a nucleic acid for gene therapy, wherein said nucleic acid encodes at least one TNF-related, molecule selected from the group consisting of intracellular TNF-induced molecules, extracellular TNF-induced molecules, TNF analogous, TNF active muteins, TNF active fragments and TNFR 2-activating molecules.

8. The method of claim 7, wherein the intracellular TNF-induced molecules are selected from the group consisting of NF-kB, MAP-K, PKB/Akt JNK, p38, erks, ask1, gck, IKK, acidic and basic Sphingomyelinases, FAN factor associated with NSMase activation, caspases and combinations thereof.

9. The method of claim 7, wherein the extracellular TNF-induced molecules are selected from the group consisting of MnSOD, GDNF, TIP-b1, BDNF, IL-6, adhesion molecules and a combinations thereof.

10. The method of claim 7, wherein the neurodegenerative disease is the Parkinson's disease.

11. The method of claim 7, wherein the treatment provides a neuroprotective effect or a delaying effect in the neurodegenerative process of the substantia nigra.

12. The method of claim 7, wherein the mammal is a human.

13. A method of treating a neurodegenerative disease in a mammal in need of such treatment, said method comprising:
i) determining the TNF levels and detecting an inflammatory disease in a group of mammals;
ii) selecting the mammals having a high TNF level and/or an inflammatory disease; and
iii) administering to the selected mammals a direct or indirect therapy that reduces the TNF levels and/or the inflammatory process.

14. The method of claim 13, wherein the therapy that reduces the TNF levels comprises administering an effective amount of at least one molecule selected from the group consisting of TNF ligand, TNF down-expression regulating molecules, TNF down-biological activity regulating molecules, methaloproteases inhibitors, TNFR 1 inactivating molecules, TNFR 1 inhibitors, and combinations thereof.

15. The method of claim 13, wherein the TNF ligand is an TNF defective receptor-binding domain; the TNF down-expression regulating molecules are selected from the group consisting of corticosteroids, anti-inflammatory cytokines; the TNF down-biological activity regulating molecules are selected from the group consisting of anti-TNF antibodies, Thalidomide, humanized anti-TNF antibody, geraniin, corilagin, epigallocatechin gallate, EtarneRcept, Infliximab, GM-CSF, histamine, Adalimumab, adrenoreceptor agonists, activators of vagal nerve activity, and antisense oligonucleotides; the methaloproteases inhibitors are selected from the group consisting of TACE inhibitors, N-terminal domain form of tissue inhibitor of metalloproteinases-3 (N-TIMP-3), CP-661,631, gamma-lactam hydroxamic acids, and E)-2(R)-[1(S)-(Hydroxycarbamoyl)-4-phenyl-3-butenyl]-2′-isobutyl-2′-(methanesulfonyl)-4-methylvalerohydrazide (Ro 32-7315); and the TNFR 1 inactivating molecules are selected from the group consisting of SODD and I-kB.

16. The method of claim 13, wherein the neurodegenerative disease is the Parkinson's disease.

17. The method of claim 13, wherein the method reduces the TNF production or induces low TNF expression for generating low TNF levels in the substantia nigra.

18. The method of claim 13, wherein the mammal is a human.

19. A method of treating a neurodegenerative disease in a mammal in need of such treatment, the method comprising:
i) determining the TNF levels and detecting an inflammatory disease in a group of mammals;
ii) selecting the mammals having a high TNF level and/or an inflammatory disease; and
iii) administering to the selected mammals a gene therapy that reduces the TNF levels or have an anti inflammatory effect.

20. The method of claim 19, wherein the therapy that reduces the TNF levels comprises administering an effective amount of a nucleic acid selected from the group consisting of nucleic acids that encodes TNF ligand, TNF down-expression regulating molecules, TNF down-biological activity regulating molecules, methaloproteases inhibitors, TNFR I inactivating molecules and TNFR I inhibitors.

21. The method of claim 19, wherein the TNF ligand is TNF defective receptor-binding domain; the TNF down-expression regulating molecules are selected from the group consisting of genes coding for anti-inflammatory cytokines, RIP, TRAF1, TRAF2, Bid, and soluble TNf receptors; the TNF down-biological activity regulating molecules are selected from the group consisting of genes coding for single chain anti-TNF antibodies, humanized anti-TNF antibody, GM-CSF, adrenoreceptor agonists, and antisense oligonucleotides; the methaloproteases inhibitors are selected from the group consisting of genes coding for TACE inhibitors, and N-terminal domain form of tissue inhibitor of metalloproteinases-3 (N-TIMP-3); and the TNFR 1 inactivating molecules are selected from the group consisting of genes coding for SODD and I-kB.

22. The method of claim 19, wherein the neurodegenerative disease is the Parkinson's disease.

23. The method of claim 19, wherein the method reduces the TNF production or induces a low TNF expression for generating low TNF levels in the substantia nigra.

24. The method of claim 19, wherein the mammal is a human.

25. A method of treating a neurodegenerative disease in a mammal in need of such treatment, said method comprising:
i) administering an effective amount of at least one TNFR 2 activating molecule and/or an effective amount of at least one compound that reduces the TNF levels.

26. The method of claim 25, wherein the at least one TNFR2 activating molecule is selected from the group consisting of NF-kB, MAP-K, PKB/Akt, MnSOD, lymphotoxin alpha, membrane TNF, receptor agonists, activating antibodies and combinations thereof.

27. The method of claim 25, wherein the neurodegenerative disease is the Parkinson's disease.

28. The method of claim 25, wherein the treatment with at least one TNFR 2 activating molecule or TNF-related molecule provides a neuroprotective effect or a delaying effect in the neurodegenerative process of the substantia nigra in mammals and the treatment with at least one TNF down regulation molecule reduces the TNF production or induces a low TNF expression for generating low TNF levels in the substantia nigra.

29. The method of claim 25, wherein the mammal is a human.

30. A method for designing a treatment schedule for treating a Parkinson's disease, the method comprising:
i) detecting the TNF levels and inflammatory diseases in a group of patients,
ii) selecting patients having a low or no TNF levels and determining a molecules dose for slightly increasing the TNF levels, wherein said molecules dose comprises molecules selected from the group consisting of TNF, TNF analogous, TNF active muteins, TNF up-regulating molecules and combinations thereof; or
iii) selecting patients having high TNF levels and determining a dose of TNF down-regulating molecules selected from the group consisting of TNF ligand, TNF down-expression regulating molecules, TNF down-activity regulating molecules, methaloproteases inhibitors, TNFR 1 inactivating molecules; TNFR 1 inhibitors and combinations thereof.

31. A. transgenic knock in mouse as a Parkinson's disease animal model, wherein at least the somatic cells of said mouse comprises a transgene comprising a DNA sequence encoding mTNF in operable linkage with the endogenous engrailed-1 promoter; and a interference cassette downstream of the transgene flanked by loxP sequences, wherein said mTNF coding sequence is expressed at low levels in the substantia nigra.

32. The transgenic knock in mouse of claim 31, wherein said mouse comprises an mTNF coding sequence under the control of a CRE/loxP switch expression system, wherein the administration of CRE recombinase to the mouse develops a Parkinson's phenotype and increases the TNF levels and mimics Parkinson's pathological conditions.

33. The transgenic knock in mouse of claim 31, wherein said endogenous engrailed-1 promoter is up-regulated by delivering an adenoviral vector that expresses a CRE recombinase in the substantia nigra.

34. A method for producing a transgenic knock in mouse as a Parkinson's disease animal model, the method comprising the steps of:
i) transferring a transgene into a mouse embryonic stem cells, wherein the transgene comprises a DNA sequence that encodes mTNF, operable linked to the endogenous engrailed-1 promoter, and a loxP interference cassette downstream or upstream of the DNA sequence;
ii) selecting an embryonic stem cell line that comprises said transgene,
iii) transferring the embryonic stem cell of step ii) into a blastocyst,
iv) transferring the blastocyst containing the transgene of step iii) into a pseudopregnant foster mother,
v) screening for a transgenic mouse born to the foster mother which includes the transgene, wherein said transgene of transgenic mouse born to the foster mother is expressed at low level in the substantia nigra.

35. A method of testing a candidate agent for treating a Parkinson's disease, the method comprising:
i) providing a first and second transgenic knock-in mouse of claim 31;
ii) injecting to said first transgenic knock-in mouse a replication-deficient adenoviral vector, wherein said adenoviral vector expresses a nuclear form of Cre recombinase, and injecting to said second transgenic knock-in mouse a control adenoviral vector;
iii) administering to said first transgenic knock-in mouse a candidate agent; and
iv) comparing Parkinson's pathological conditions of said first transgenic knock-in mouse of step iii) to the Parkinson's disease parameters of said second transgenic knock-in mouse, wherein the reduction in Parkinson's pathological conditions in said first transgenic knock-in mouse indicates that said administrated candidate agent is potentially useful for Parkinson's diseases treatment.

36. The use of TNF-agonist molecules in the manufacture of a medicament for a Parkinson's disease treatment, wherein said TNF-related molecules are selected from the group consisting of intracellular TNF-induced molecules, extracellular TNF-induced molecules, TNF analogous, TNF active muteins, TNF active fragments and TNFR 2 activating molecules, and wherein the administration of a pharmaceutically effective amount of said medicament induces a neuroprotective effect.

37. The use of TNF down regulating molecules in the manufacture of a medicament for a Parkinson's disease treatment, wherein said TNF down regulating molecules are selected from the group consisting of TNF ligand, TNF down-expression regulating molecules, TNF down-biological activity regulating molecules, methaloproteases inhibitors, TNFR 1 inactivating molecules and TNFR 1 inhibitors; and wherein the administration of a pharmaceutically effective amount of said medicament reduces the TNF levels.
